(19) United States
(12) Patent Application Publication
(10) Pub. No.: US 2011/0129463 A1
(43) Pub. Date: Jun. 2, 2011

(54) Quinazolin-4(3A)-One Derivatives and Methods of Use Thereof

(75) Inventors: Omri EREZ, Rehovot (IL); Philippe NAKACHE, Nes Ziona (IL)

(73) Assignee: Proteologics, Ltd., Rehovot (IL)

(21) Appl. No.: 12/954,793

(22) Filed: Nov. 26, 2010

Related U.S. Application Data
(60) Provisional application No. 61/264,741, filed on Nov. 27, 2009.

(51) Int. Cl. A61K 31/517; A61P 35/00; A61P 29/00; A61P 37/06; A61P 35/02; A61K 31/506; A61K 31/496; A61K 31/5377; A61K 31/553; A61K 31/675; A61K 31/7072; A61K 31/7048; A61K 38/50; A61K 38/43; A61K 33/24; A61P 3/10; A61P 11/00; A61P 9/10; A61P 19/04; A61P 13/12; A61P 11/06; A61P 17/00; A61P 25/28; A61P 19/02; A61P 7/06; A61P 27/14; A61P 1/00; A61K 31/56; A61K 39/395; C07D 239/88

(52) U.S. Cl. 424/133.1; 514/266.3; 514/266.23; 514/252.18; 514/252.19; 514/253.06; 514/234.5; 514/211.08; 514/90; 514/50; 514/27; 424/94.6; 424/94.1; 424/649; 514/171; 544/287

(57) ABSTRACT

Provided are quinazolin-4(3A)-one derivatives, which are inhibitors of the ubiquitin ligase activity of a human polypeptide, particularly to POSH inhibitors, and to compositions and methods for the treatment RING E3 ubiquitin ligase related diseases.

### FIELD

The presently described subject matter relates to small pyrimidine derivatives, which are inhibitors of the ubiquitin ligase activity of a human polypeptide, particularly to POSH inhibitors, and to compositions and methods for the treatment RING E3 ubiquitin ligase related diseases.

### BACKGROUND

Potential drug target validation involves determining whether a DNA, RNA or protein molecule is implicated in a disease process and is therefore a suitable target for development of new therapeutic drugs. Drug discovery, the process by which bioactive compounds are identified and characterized, is a critical step in the development of new treatments for human diseases. The landscape of drug discovery has changed dramatically due to the genomics revolution. DNA and protein sequences are yielding a host of new drug targets and an enormous amount of associated information.

The identification of genes and proteins involved in various disease states or key biological processes, such as cancer, inflammation and immune response, is a vital part of the drug design process. Many diseases and disorders could be treated or prevented by decreasing the expression of one or more genes involved in the molecular etiology of the condition if the appropriate molecular target could be identified and appropriate antagonists developed. For example, cancer, in which one or more cellular oncogenes become activated and result in the unchecked progression of cell cycle processes, could be treated by antagonizing appropriate cell cycle control genes. Furthermore many human genetic diseases, such as Huntington's disease, and certain prion conditions, which are influenced by both genetic and epigenetic factors, result from the inappropriate activity of a polypeptide as opposed to the complete loss of its function. Accordingly, antagonizing the aberrant function of such mutant genes would provide a means of treatment. Drug therapy strategies for treating such diseases and disorders have frequently employed molecular antagonists which target the polypeptide product of the disease gene(s). However the discovery of relevant gene or protein targets is often difficult and time consuming.

Targeted proteins undergoing selective degradation, presumably through the actions of an ubiquitin-dependent protaeosome, are covalently tagged with ubiquitin through the formation of an isopeptide bond between the C-terminal glycyl residue of ubiquitin and a specific lysyl residue in the substrate protein.

Ubiquitin is attached to proteins by a cascade of enzymes comprising ubiquitin-activating enzyme (E1), ubiquitin-conjugating enzymes (E2s) and ubiquitin ligases (E3s) (Hershko et al., 1983). E1 binds to and activates ubiquitin and then transfers it to an E2 enzyme. Subsequently, E3-ubiquitin ligases play a critical role in the final step of the ubiquitination process by recruiting ubiquitin linked E2s, recognizing specific substrates, and mediating, or directly catalyzing, ubiquitin transfer to the substrates. E3 ubiquitin ligases can be classified into several sub-families; The RING domain-containing E3s, the HECT domain-containing E3s, the U box and the PHD domain-containing E3s.

The RING domain subfamily is the largest subfamily containing hundreds of different E3s. RING E3s facilitates the direct transfer of ubiquitin from E2 enzymes to the substrates. The ubiquitin system of protein modification is a crucial mechanism involved in almost every aspect of cellular processes. The evidence for the involvement of the ubiquitin system in human diseases is rapidly accumulating, thus reflecting the central role of the system in cellular function. The lack of classic enzymatic activity in the mode of action of the RING E3s make the task of targeting a specific E3 by small-molecular weight inhibitors for therapeutics uses very challenging.

POSH is an E3 ubiquitin ligase that was first identified as Rac1 target involved in signal transduction pathways leading to activation of the INK pathway, nuclear translocation of NF-kappaB and induction of apoptosis (Tapon et al., 1998). The vast majority of the reports linked POSH to pre-apoptotic function by acting as a scaffold for components of the JNK pathway such as mixed-lineage kinases (MLKs), MAP kinase kinases (MKKs) 4 and 7, c-Jun N-terminal kinases (JNKs) (Aigaki et al., 2002; Figueroa et al., 2003; Kim et al., 2005a; Kukekov et al., 2006; Seong et al., 2001; Tapon et al., 1998; Tsuda et al., 2005; Tsuda et al., 2006; Wang et al., 2007; Wilhelm et al., 2007; Xu et al., 2003; Xu et al., 2005; Xu et al., 2006; Zhang et al., 2005). Only few reports implicated POSH in other functions. Another report suggested that POSH is a trans-Golgi network-associated protein involved in the targeting of HIV-1 to the plasma membrane (Alroy et al., 2005). Another possible link to virus release is a report that describes that POSH forms a complex with Apoptosis-linked gene-2 (ALG-2) and ALG-2-interacting protein (ALIX/AIP1) in a calcium-dependent manner in Drosophila (Tsuda et al., 2006). Alix is a key player in vesicle formation at the multivesicular body and involved in virus budding (Fujii et al., 2007). It was suggested that POSH/ALG-2/ALIX complex might function together in the regulation of the JNK pathway (Tsuda et al., 2006). POSH also regulates calcium homeostasis in the ER through ubiquitination of Herp (Tuvia et al., 2007) and ubiquitinates hepatocyte growth factor-regulated tyrosine kinase substrate (Hrs) on early endosomes (Kim et al., 2005b). Recently it was reported that POSH also regulates axon outgrowth (Taylor et al., 2008). It was shown that silencing of POSH by RNAi enhances axon outgrowth in mouse cortical neurons and in neurons derived from mouse P19 cells. POSH activity was mediated by actomyosin regulatory protein Shroom3 that recruits Rho kinase to inhibit process outgrowth.

### SUMMARY

It was found, according to the presently described subject matter, that certain pyrimidine derivatives act as RING E3 ubiquitin ligase inhibitors, and are capable of blocking cell migration.

The presently described subject matter is directed to small-molecules which strongly inhibit RING E3 ubiquitin ligase activity. The present subject matter further describes the potential of inhibitory molecules as therapeutic agents for different indications involving RING E3 ubiquitin ligase related diseases. Such RING E3 ubiquitin ligase related diseases, include but are not limited to, cell migration diseases, disorders or conditions including, for example cancer, different inflammation-related disorders, and angiogenesis-related disorders such as age-related macular degeneration (AMD) and retinopathies, and genetic disorders.

In an embodiment, the presently described subject matter is directed to a method for treating a RING E3 ubiquitin ligase related disease in a subject, comprising administering to the subject a therapeutically effective amount of a presently described compound.

In another embodiment, the presently described subject matter is directed to a method for treating a RING E3 ubiquitin ligase related disease in a subject, comprising administering to the subject a therapeutically effective amount of a pharmaceutical composition comprising the presently described compounds.

### BRIEF DESCRIPTION OF THE FIGURES

FIG. 1 shows the characterization of compound A by HNMR.

FIG. 2A is a graphical representation of the killing abilities of compound A, B, C and D in A375 cancer cells.

FIG. 2B is a graphical representation of the cell migration inhibition abilities of compound A in A375 cancer cells.

FIG. 2C shows the inhibitory effect of Compound B on cell-migration with IC50 of 0.045 μM in A375 melanoma cell line.

FIG. 2D shows the inhibitory effect of Compound C on cell-migration with IC50 of 0.92 μM in A375 melanoma cell line.

FIG. 2E shows the inhibitory effect of Compound D on cell-migration with 1050 of 0.061 μM in A375 melanoma cell line.

FIG. 3 is a graphical representation of the in-vivo effect of compound A against formation of lung cancer metastasis.

FIG. 4 is a graphical representation of the suppressive activity of compound A in an EAE model.

FIG. 5 shows the characterization of compound B by HNMR.

FIG. 6 shows the characterization of compound C by HNMR.

FIG. 7 shows a DTH study with Compounds A, B and C.

### DETAILED DESCRIPTION

### Definitions

Without limiting the scope to further possible definitions, as used herein in the specification, the terms hereinbelow are defined as follows:

The term “Alkyl” refers to a hydrocarbon chain that may be a straight chain or branched chain, containing the indicated number of carbon atoms. For example, C₁-C₁₀ indicates that the group may have from 1 to 10 (inclusive) carbon atoms in it.

The “alkyl”, “alkenyl”, or “alkynyl” radical is a “C₁-C₁₀ alkyl,” or a “C₁-C₆ alkyl,” a “C₁-C₁₂ alkenyl,” “C₂-C₁₂ alkenyl,” or a “C₁-C₆ alkenyl,” a “C₁-C₁₂ alkynyl,” “C₂-C₁₂ alkenyl,” or a “C₁-C₆ alkynyl,” respectively, that may be straight or branched chain.

The term “C₁-C₆ alkyl” refers to a straight or branched chain saturated hydrocarbon containing 1-6 carbon atoms. Examples of a C₁-C₆ alkyl group include, but are not limited to, methyl, ethyl, propyl, isopropyl, n-pentyl, isopentyl, neopentyl, and hexyl.

The term “alkenyl” refers to a straight or branched chain unsaturated hydrocarbon.

The term “C₁-C₆ alkenyl” refers to a straight or branched chain unsaturated hydrocarbon containing 1-6 carbon atoms and at least one double bond. Examples of a C₁-C₆alkenyl group include, but are not limited to, methylene, ethylene, propylene, 1-butylene, 2-butylene, isobutylene, sec-butylene, 1-pentene, 2-pentene, isopentene, 1-hexene, 2-hexene, 3-hexene, and isohexene.

The term “C₂-C₁₂ alkenyl” refers to a straight or branched chain unsaturated hydrocarbon containing 2-12 carbon atoms and at least one double bond.

The term “C₁-C₁₀ alkoxy” refers to a straight or branched chain saturated or unsaturated hydrocarbon containing 1-10 carbon atoms and at least one oxygen atom.

The term “alkoxyl” refers an alkyl group linked to oxygen thus: R—O.

The term “amino group” refers to a functional group that consists of a nitrogen atom attached by single bonds to hydrogen atoms.

The term “Aryl” refers to cyclic aromatic carbon ring systems containing from 6 to 18 carbons. Examples of an aryl group include, but are not limited to, phenyl, naphthyl, anthracenyl, tetracenyl, and phenanthrenyl.

The term “arylalkyl” refers to an aryl group with at least one alkyl substitution. Examples of arylalkyl include, but are not limited to, toluenyl, phenylethyl, xylenyl, phenylbutyl, phenylpentyl, and ethylnaphthyl.

The term cyano refers to C≡N.

The term cycloalkenyl,” respectively, namely, 5-10 partially unsaturated carbocyclic groups and include, for example, cyclopentenyl and cyclohexenyl.

The term “Halogen” refers to an atom of fluorine, chlorine, bromine, or iodine.

The term “Heteroaryl” refers to mono and bicyclic aromatic groups of 4 to 10 atoms containing at least one heteroatom. Heteroatom as used in the term heteroaryl refers to oxygen, sulfur and nitrogen. Examples of monocyclic heteroaryls include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, and pyrimidinyl.

The term “Heteroarylalkyl” refers to a heteroaryl group with at least one alkyl substitution.

The term “hydroxyl” refers to the functional group —OH.

The term “nitro” refers to a functional group having a nitrogen atom joined to two oxygen atoms.

The term “heteroatom” means an atom selected from N, S and/or O.

The term “aryl” refers to a “C₆-C₁₄” aromatic carbocyclic group having 6 to 14 carbon atoms or 6 to 10 carbon atoms, consisting of a single, bicyclic or tricyclyc ring, such as phenyl, naphthyl and antracenyl, that may be substituted by one or more radicals as defined herein above.

The term “heterocyclyl” means a radical derived from saturated or partially unsaturated (non-aromatic) monocyclic, bicyclic or tricyclic heterocycle, of 2 to 8, or 5 to 6, ring members, of which ring members one to three is a heteroatom selected from O, S and/or N. Non-limiting examples of non-aromatic heterocyclyls include dihydrofuryl, tetrahydrofuryl, dihydrothienyl, pyrrolydinyl, pyrrolynyl, dihydropyridyl, piperidinyl, piperazinyl, morpholino, and the like.

The term “heteroaryl” as used herein, means a radical derived from a mono- or poly-cyclic heteroaromatic ring containing one to three heteroatoms selected from the group consisting of O, S and N. Particular examples are pyrrolyl, furyl, thienyl, pyrazolyl, imidazolyl, oxazolyl, thiazolyl, pyridyl, quinolinyl, isoquinolinyl, pyridazinyl, pyrimidinyl, pyrazinyl, 1,3,4-triazinyl, 1,2,3-triazinyl, 1,3,5-triazinyl, benzofuryl, isobenzofuryl, indolyl, imidazo[1,2-a]pyridyl, benzimidazolyl, benzthiazolyl and benzoxazolyl, benzodiazepinyl, and other radicals derived from further polycyclic heteroaromatic rings. The heteroaryl radical may be substituted by one or more radicals as defined herein above. It is to be understood that when a polycyclic heteroaryl ring is substituted, the substitutions may be in any of the carbocyclic and/or heterocyclic rings. In one embodiment the heteroaryl is thienyl.

The term “halogen” refers to fluoro, chloro, bromo or iodo. In some embodiments, the halogen is chloro.

As used herein, the terms “administering,” “administration,” and like terms refer to any method which, in sound medical practice, delivers a composition to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The phrase “angiogenesis-related disorders” means any angiogenesis-related condition involving the growth or undesired growth of new blood vessels. Such angiogenesis-related conditions can include conditions related to excessive or undesired angiogenesis such as diabetic blindness; chronic inflammation; arthritis; age-related macular degeneration; retinopathy; rheumatoid arthritis; osteoarthritis; Crohn's disease; psoriasis; cancer; Alzheimer's disease; restenosis; pulmonary fibrosis; asthma; angiofibroma; neovascular glaucoma; arteriovenous malformations; nonunion fractures; lupus and other connective tissue disorders; Osler-Weber syndrome; atherosclerotic plaques; corneal graft neovascularization; Pyogenic granuloma; retrolental fibroplasias; scleroderma; granulations, hemangioma; trachoma; hemophilic joints; peritoneal endometriosis; adiposity; and vascular adhesions.

The phrase “angiogenesis inhibitor” means any substance that inhibits angiogenesis. Suitable angiogenesis inhibitors include, but are not limited to, bevacizumab (AVASTINT™), sunitinib (SUTENT™), sorafenib (NEXAVAR™), thalidomide (THALOMID™), lenalidomide (REVLIMID™), panitumumab (VECTIBIX™), cetuximab (ERBITUX™), and erlotinib (TARCEVAT™).

The phrase “anti-cancer agent” means one or more tyrosine kinase inhibitors. Suitable tyrosine kinase inhibitors include, but are not limited to, imatinib, dasatinib, axitinib, bosutinib, cediranib, erlotinib, gefitinib, lapatinib, lestaurtinib, nilotinib, semaxanib, sutinib, toceranib, vandetanib, and vatalanib.

The phrase “anti-inflammatory agent” means any substance that reduces or suppresses inflammation. Suitable anti-inflammatory agents include, but are not limited to, corticosteroid including for example cortisol, aldosterone, hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, flucinonide, fluocinolone acetonide, halcinonide, betamethasone, betamethasone sodium phosphate, dexamethasone, dexamethasone dodium phosphate, flucortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, flucortolone caproate, fluocortolone pivalate, and fluprednidene acetate; a non-steroidal anti-inflammatory drug including for example, a cox-2 inhibitor, nimesulide, diclofenac, licofelone, aspirin, ibuprofen, and naproxen; immune selective anti-inflammatory derivatives such as phenylalanine-glutamine-glycine and its D-isomeric form; and herbs including for example, Harpagophytum, hyssop, ginger, turmeric, Arnica montana, willow bark, and cannabis.

The term “cancer” is the generic name of a group of diseases that are characterised by abnormal, uncontrolled cell division, the ability to invade normal tissues and the ability to spread to other parts of the body. Such cancers can include anal cancer; astrocytoma; leukemia; lymphoma; head and neck cancer; liver cancer; testicular cancer; cervical cancer; sarcoma; hemangioma; esophageal cancer; eye cancer; laryngeal cancer; mouth cancer; mesothelioma; myeloma; oral cancer; rectal cancer; throat cancer; bladder cancer; breast cancer; uterine cancer; ovarian cancer; prostate cancer; lung cancer; colon cancer; pancreatic cancer; renal cell carcinoma; gastric cancer; skin cancer; including basal cell carcinoma, melanoma, and squamous cell carcinoma; oral squamous cell carcinoma; colorectal cancer; glioblastoma multiforme; endometrial cancer; and malignant glioma.

The phrase “cancer chemotherapeutic agent” means any known chemotherapeutic agent that may be used for combination therapy with the presently described subject matter, and includes, but is not limited to alkylating agents, including mechlorethamine, cyclophosphamide, ifosfamide, melphalan, chlorambucil, dicarbazine, streptazocine, carmustine, lomustine, semustine, chlorozotocin, busulfan, triethylenemelamine, thiotepa, hexamethylmelamine; antimetabolites, including methotrexate; fluorouracil; 5-fluorouracil; floxuridine (5′-fluoro-2′-deoxyuridine); idoxuridine; cytarabine; N-phosphonoacetyl-L-aspartate; 5-azacytidine; azaribine; 6-azauridine; pyrazofuran; 3-deazauridine; acivicin; purine analogs, including thioguanine, mercaptopurine, azathioprine, pentostatin, erythrohydroxynonyladenine; vinca alkaloids, including vincristine and vinblastine; epipodophyllotoxins, including etoposide and teniposide; antibiotics, including dactinomycin, daunorubicin, doxorubicin, bleomycin sulfate, plicamycin, mitomycin; enzymes, including L-asparaginase; platinum coordination complexes, including cisplatin, carboplatin; hydroxyurea, procarbazine, mitotane; and hormones or related agents, including adrenocorticosteroids such as prednisone and prednisolone; aminoglutethimide; progestins such as hydroxyprogesterone caproate, medroxyprogesterone acetate, megesterol acetate, estrogens and androgens such as diethylstilbestrol, fluoxymesterone, ethynyl estradiol, antiestrogens such as tamoxifen, and gonadotropin-releasing hormone analogs such as leuprolide.

In practicing the presently described methods for treating cancer, the present compounds may be administered together with at least one known chemotherapeutic agent and/or at least one anti-cancer agent as part of a unitary pharmaceutical composition. Alternatively, the present compounds may be administered apart from at least one known cancer chemotherapeutic agent and/or at least one anti-cancer agent. In one embodiment, the present compounds and at least one known cancer chemotherapeutic agent and/or at least one anti-cancer agent are administered substantially simultaneously, i.e. the compounds are administered at the same time or one after the other, so long as the compounds reach therapeutic levels in the blood at the same time. In another embodiment, the present compounds and at least one known cancer chemotherapeutic agent and/or at least one anti-cancer agent are administered according to their individual dose schedule, so long as the compounds reach therapeutic levels in the blood.

Similarly, another embodiment of the presently described subject matter is directed to a method of treating cancer by administering the presently described compounds in combination with radiation therapy. In this embodiment, the presently described compounds may be administered at the same time as the radiation therapy is administered or at a different time.

The phrase “disease, disorder or condition related to cell migration” means any disease, disorder or condition where aberrant cell migration is a feature. Such diseases, disorders or conditions can include cancer; different inflammation-related disorders including autoimmune diseases; and angiogenesis-related disorders such as age-related macular degeneration (AMD), retinopathies and others.

As used herein, the term “carrier,” “excipient,” or “adjuvant” refers to any component of a pharmaceutical composition that is not the drug substance.

As used herein, the phrases “drug product,” “pharmaceutical dosage form,” “final dosage form,” and the like, refer to the combination of one or more drug substances and one or more excipients (i.e., pharmaceutical composition) that is administered to a patient in need of treatment, and can be in the form of a solution, an aqueous solution, an emulsion, a suspension, tablets, capsules, patches, suppositories, a cream, a gel, a lotion, and the like.

Pharmaceutical compositions containing a presently described compound or its pharmaceutically acceptable salts as the active ingredient can be prepared according to conventional pharmaceutical compounding techniques. See, for example, Remington's Pharmaceutical Sciences, 18th Ed. (1990, Mack Publishing Co., Easton, Pa.).

The phrase “inflammation-related disorders” means any inflammatory condition where aberrant cell migration is a feature. Such inflammatory conditions can include pulmonary fibrosis; ischaemic heart disease; autoimmune diseases such as Crohn's disease, dermatomyositis, diabetes mellitus, Guillain-Barre syndrome, hashimoto's disease, idiopathic thrombocytopenic purpura, mixed connective tissue disease, myasthenia gravis, narcolepsy, pemphigus vulgaris, pernicious anaemia, polymyositis, primary biliary cirrhosis, Sjogren's syndrome, temporal arteritis, ulcerative colitis, vasculitis, Wegener's granulomatosis, systemic lupus erythematosus, lupus nephritis, Goodpasture's syndrome, haemolytic anaemia, thyrotoxicosis, multiple sclerosis, and scleroderma; chronic inflammatory conditions such as asthma, rheumatoid arthritis, osteoarthritis, septicaemia, artherosclerosis, chronic renal disease, inflammatory bowel disease, and vasculitis; peritonitis; ocular inflammatory diseases such as giant papillary conjunctivitis, uveitis, and seasonal allergic conjunctivitis; chronic prostatitis; glomerulonephritis; hypersensitivities; inflammatory bowel diseases; pelvic inflammatory disease; reperfusion injury; transplant rejection; Chediak-Higashi syndrome; chronic granulomatous disease; urinary tract inflammatory conditions; interstitial cystitis; ulcerative colitis; systemic sclerosis; dermatomyositis; polymyositis, and inclusion body myositis.

As used herein, the term “pharmaceutically acceptable carrier” refers to a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, tabletting agents, stabilizers, antioxidants, and preservatives may also be present.

Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO.

These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 18th Ed., Mack Publishing Co., Easton, Pa. (1990), both of which are incorporated by reference herein in their entirety.

The carrier may comprise, in total, from about 0.1% to about 99.99999% by weight of the pharmaceutical compositions presented herein.

The presently described pharmaceutical compositions herein can be in the form of oral compositions. The oral compositions contemplated herein may take the form of tablets, capsules, soft-gels, hard gels, solutions, suspensions, powders, dispersible granules, cachets, combinations thereof, or any other oral pharmaceutical dosage form as would commonly be known in the art.

A solid carrier can be one or more substances which may also act as diluents, flavoring agents, solubilizers, lubricants, suspending agents, binders, or tablet disintegration agents; it can also be an encapsulating material. In powders, the carrier can be a finely divided solid which is in admixture with the active compound. In a tablet, the active compound can be mixed with a carrier having the necessary binding properties in suitable proportions and compacted in the size and shape desired. Non-limiting examples of suitable solid carriers include magnesium carbonate, magnesium stearate, talc, cornstarch, sugar, lactose, pectin, dextrin, starch, gelatin, tragacanth, methyl cellulose, sodium carboxymethylcellulose, hydroxypropyl methylcellulose, hydroxyethylcellulose, hydroxypropylcellulose, other cellulose derivatives, a low melting wax, cocoa butter, and the like.

As used herein, the phrase “pharmaceutically acceptable salts” refers to salts of certain ingredient(s) which possess the same activity as the unmodified compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, and naturally and synthetically derived amino acids.

If organic bases are used, poorly volatile bases are preferably employed, for example low molecular weight alkanolamines such as ethanolamine, diethanolamine, N-ethylethanolamine, N-methyldiethanolamine, triethanolamine, diethylaminoethanol, 2-amino-2-methyl-n-propanol, dimethylaminopropanol, 2-amino-2-methylpropanediol, and triisopropanolamine. Ethanolamine is suitable in this regard. Further poorly volatile bases which may be mentioned are, for example, ethylenediamine, hexamethylenediamine, morpholine, piperidine, piperazine, cyclohexylamine, tributylamine, dodecylamine, N,N-dimethyldodecylamine, stearylamine, oleylamine, benzylamine, dibenzylamine, N-ethylbenzylamine, dimethylstearylamine, N-methylmorpholine, N-methylpiperazine, 4-methylcyclohexylamine, and N-hydroxyethylmorpholine.

Salts of quaternary ammonium hydroxides such as trimethylbenzylammonium hydroxide, tetramethylammonium hydroxide, or tetraethylammonium hydroxide can also be used, as can guanidine and its derivatives, in particular its alkylation products. However, it is also possible to employ as salt-forming agents, for example, low molecular weight alkylamines such as methylamine, ethylamine, or triethylamine. Suitable salts for the components to be employed according to the present subject matter are also those with inorganic cations, for example alkali metal salts, in particular sodium, potassium, or ammonium salts, alkaline earth metal salts such as, in particular, the magnesium or calcium salts, as well as salts with bi- or tetravalent cations, for example the zinc, aluminum, or zirconium salts. Also contemplated are salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Aqueous or oil-soluble or dispersible products are thereby obtained.

As used herein, the terms “POSH,” “POSH protein(s)” or “POSH polypeptide(s)” are used interchangeably and refer to a polypeptide that includes in its amino acid sequence a RING domain and at least one SH3 domain. In some instances, the POSH protein may have 3 or 4 SH3 domains.

The terms “POSH-mediated ubiquitination” or “POSH protein-mediated ubiquitination” are used interchangeably and refer to any ubiquitination process that requires the involvement of a POSH protein.

The terms “ubiquitination inhibitor,” “POSH inhibitor” or “POSH protein inhibitor” are used interchangeably and refer to a pyrimidine derivative of formula I herein that inhibits a POSH activity as defined in PCT/US02/36366 (WO 03/095972), hereby incorporated herein by reference in its entirety as if fully disclosed herein, including POSH protein-mediated ubiquitination.

The active agent is preferably administered in a therapeutically effective amount. As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the presently described manner. The phrase “therapeutically effective amount” as used herein refers to an amount of the presently described active agent effective to yield a desired therapeutic response. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

As used herein, the terms “subject” or “individual” or “animal” or “patient” or “mammal,” refers to any subject, particularly a mammalian subject, for whom diagnosis, prognosis, or therapy is desired, for example, a human.

As used herein, the terms “treatment” or “treating” of a disease, disorder, or condition encompasses alleviation of at least one symptom thereof, a reduction in the severity thereof, or the delay, prevention, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, provide improvement to a patient or subject's quality of life, or delay, prevent, or inhibit the onset of a disease, disorder, or condition.

The phrase “RING E3 ubiquitin ligase related disease” refers to any disease, disorder or condition that can be treated by inhibiting or modulating ubiquitin ligase activity. Such diseases include but are not limited to, cell migration diseases, disorders and conditions, including for example, cancer, angiogenesis disorders, and inflammatory disorders; genetic disorders including Huntington's disease, Parkinson's disease, Lafora disease (LD), Angelman syndrome, Von Hippel-Lindau syndrome, Liddle's syndrome, Fanconi anemia, and 3-M syndrome; and renal disease.

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or thickness, are to be understood to include any integer within the recited range, unless otherwise indicated.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

Throughout the application, descriptions of various embodiments use “comprising” language; however, it will be understood by one of skill in the art, that in some specific instances, an embodiment can alternatively be described using the language “consisting essentially of” or “consisting of.”

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Other terms as used herein are meant to be defined by their well-known meanings in the art.

An embodiment of the presently described subject matter is directed to a method for treating a RING E3 ubiquitin ligase related disease in a subject, comprising administering to the subject a therapeutically effective amount of a compound of general formula I

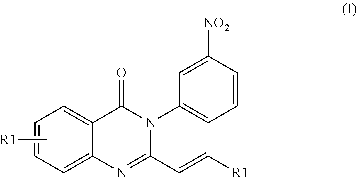

wherein:

R1 is H, C₁₋₆ alkyl, C₁₋₆ alkenyl, C₁₋₆ alkynyl, arylalkyl, cyano, hydroxyl, alkoxyl, hydroxyalkyl, or alkoxyalkyl, aryl or heteroaryl, optionally substituted by 1-5 R₃;

each R₃ is independently selected from halogen, hydroxy, hydroxyalkyl, hydroxyalkoxy, C₁-C₁₀ alkyl, C haloalkyl, alkoxy, alkoxyalkyl, alkoxyalkoxy, C₁-C₆ haloalkoxy, C₆-C₁₀ aryl, C₄-C₁₀ heteroaryl, C₇-C₁₂ aralkyl, C₅-C₁₂ heteroaralkyl, C₂-C₈ heterocyclyl, C₂-C₁₂ alkenyl, C₂-C₁₂ alkynyl, C₅-C₁₀ cycloalkenyl, C₄-C₁₀ heterocycloalkenyl, carboxy, carboxylate, cyano, nitro, amino, aminoalkyl, C₁-C₆ alkylamino, C₁-C₆ alkylaminoalkyl, C₁-C₆ dialkylamino, C₁-C₆ dialkylaminoalkyl, mercapto, SO₃H, —S— alkyl, —S(O)alkyl, S(O)₂alkyl, sulfate, S(O)NH₂, S(O)₂NH₂, phosphonyl, acyl, aminocarbonyl, C₁-C₆ alkylaminocarbonyl, C₁-C₆ dialkylaminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, alkoxyaminocarbonyl, —NHSO₂NH₂, or —OCH₂CH₂N(R₇)₂;

R2 is H, C₁₋₆ alkyl, C₁₋₆ alkenyl, C₁₋₆ alkynyl, arylalkyl, cyano, hydroxyl, alkoxyl, hydroxyalkyl, or alkoxyalkyl, aryl or heteroaryl, optionally substituted by 1-5 R₃;

each R₃ is as described above;

or an enantiomer or pharmaceutically acceptable salt thereof.

In another embodiment, the presently described subject matter is directed to a method for treating a RING E3 ubiquitin ligase related disease in a subject, comprising administering to the subject a therapeutically effective amount of a pharmaceutical composition comprising a compound of general formula I

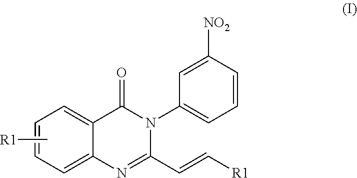

wherein:

R1 is H, C₁₋₆ alkyl, C₁₋₆ alkenyl, C₁₋₆ alkynyl, arylalkyl, cyano, hydroxyl, alkoxyl, hydroxyalkyl, or alkoxyalkyl, aryl or heteroaryl, optionally substituted by 1-5 R₃;

each R₃ is independently selected from halogen, hydroxy, hydroxyalkyl, hydroxyalkoxy, C₁-C₁₀ alkyl, C₁-C₆ haloalkyl, C₁-C₁₀ alkoxy, alkoxyalkyl, alkoxyalkoxy, C₁-C₆ haloalkoxy, C₆-C₁₀ aryl, C₄-C₁₀ heteroaryl, C₇-C₁₂ aralkyl, C₅-C₁₂ heteroaralkyl, C₂-C₈ heterocyclyl, C₂-C₁₂ alkenyl, C₂-C₁₂ alkynyl, C₅-C₁₀ cycloalkenyl, C₄-C₁₀ heterocycloalkenyl, carboxy, carboxylate, cyano, nitro, amino, aminoalkyl, C₁-C₆ alkylamino, C₁-C₆ alkylaminoalkyl, C₁-C₆ dialkylamino, C₁-C₆ dialkylaminoalkyl, mercapto, SO₃H, —S— alkyl, —S(O)alkyl, S(O)₂alkyl, sulfate, S(O)NH₂, S(O)₂NH₂, phosphonyl, acyl, aminocarbonyl, C₁-C₆ alkylaminocarbonyl, C₁-C₆ dialkylaminocarbonyl, C₁-C₁₀ alkoxycarbonyl, thioalkoxycarbonyl, hydrazinocarbonyl, C₁-C₆ alkyl hydrazinocarbonyl, C₁-C₆ dialkyl hydrazinocarbonyl, hydroxyaminocarbonyl, alkoxyaminocarbonyl, —NHSO₂NH₂, or —OCH₂CH₂N(R₇)₂;

R2 is H, C₁₋₆ alkyl, C₁₋₆ alkenyl, C₁₋₆ alkynyl, arylalkyl, cyano, hydroxyl, alkoxyl, hydroxyalkyl, or alkoxyalkyl, aryl or heteroaryl, optionally substituted by 1-5 R₃;

each R₃ is as described above;

or an enantiomer or pharmaceutically acceptable salt thereof.

In a further embodiment, the presently described subject matter is directed to a method for treating a RING E3 ubiquitin ligase related disease, wherein the compound is:

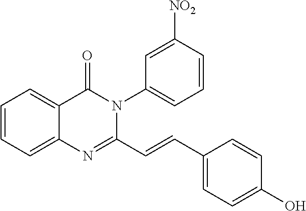

In yet a further embodiment, the presently described subject matter is directed to a method for treating a RING E3 ubiquitin ligase related disease, wherein the pharmaceutical composition further comprises at least one pharmaceutically acceptable carrier.

In another embodiment, the presently described subject matter is directed to a method for treating a RING E3 ubiquitin ligase related disease, wherein the RING E3 ubiquitin ligase related disease is selected from the group consisting of cancer, an inflammatory disorder, an autoimmune disease, and an angiogenesis disorder.

In another embodiment, the presently described subject matter is directed to a method for treating a RING E3 ubiquitin ligase related disease, wherein the RING E3 ubiquitin ligase related disease is cancer.

In still another embodiment, the presently described subject matter is directed to a method for treating cancer, wherein the cancer is selected from the group consisting of anal cancer; astrocytoma; leukemia; lymphoma; head and neck cancer; liver cancer; testicular cancer; cervical cancer; sarcoma, hemangioma; esophageal cancer; eye cancer; laryngeal cancer; mouth cancer; mesothelioma; myeloma; oral cancer; rectal cancer; throat cancer; bladder cancer; breast cancer; uterine cancer; ovarian cancer; prostate cancer; lung cancer; colon cancer; pancreatic cancer; renal cell carcinoma; gastric cancer; skin cancer; basal cell carcinoma; melanoma; squamous cell carcinoma; oral squamous cell carcinoma; colorectal cancer; glioblastoma multiforme; endometrial cancer; and malignant glioma.

In an embodiment, the presently described subject matter is directed to a method for treating cancer, wherein the pharmaceutical composition further comprises an effective amount of at least one anti-cancer agent.

In a further embodiment, the presently described subject matter is directed to a method for treating cancer, wherein the at least one anti-cancer agent is selected from the group consisting of imatinib, dasatinib, axitinib, bosutinib, cediranib, erlotinib, gefitinib, lapatinib, lestaurtinib, nilotinib, semaxanib, sunitinib, toceranib, vandetanib, and vatalanib.

In yet a further embodiment, the presently described subject matter is directed to a method for treating cancer, further comprising administering to the subject an effective amount of at least one anti-cancer agent.

In still a further embodiment, the presently described subject matter is directed to a method for treating cancer, wherein the at least one anti-cancer agent is administered simultaneous with, before or after administration of the pharmaceutical composition.

In an embodiment, the presently described subject matter is directed to a method for treating cancer, wherein the pharmaceutical composition further comprises an effective amount of at least one cancer chemotherapeutic agent.

In another embodiment, the presently described subject matter is directed to a method for treating cancer, wherein the at least one cancer chemotherapeutic agent is selected from the group consisting of mechlorethamine; cyclophosphamide; ifosfamide; melphalan; chlorambucil; dicarbazine; streptazocine; carmustine; lomustine; semustine; chlorozotocin; busulfan; triethylenemelamine; thiotepa; hexamethylmelamine; an antimetabolite; methotrexate; fluorouracil; 5-fluorouracil; floxuridine (5-fluoro-T-deoxyuridine); idoxuridine; cytarabine; N-phosphonoacetyl-L-aspartate; 5-azacytidine; azaribine; 6-azauridine; pyrazofuran; 3-deazauridine; acivicin; a purine analog; thioguanine; mercaptopurine; azathioprine; pentostatin; erythrohydroxynonyladenine; a vinca alkaloid; vincristine; vinblastine; an epipodophyllotoxin; etoposide; teniposide; an antibiotic; dactinomycin; daunorubicin; doxorubicin; bleomycin sulfate; plicamycin; mitomycin; an enzyme; L-asparaginase; a platinum coordination complex; cisplatin; carboplatin; hydroxyurea; procarbazine; mitotane; a hormone; an adrenocorticosteroid; prednisone; prednisolone; aminoglutethimide; a progestin; hydroxyprogesterone caproate; medroxyprogesterone acetate; megesterol acetate; estrogen; an androgen; diethylstilbestrol; fluoxymesterone; ethynyl estradiol; an antiestrogen; tamoxifen; a gonadotropin-releasing hormone analog; and leuprolide.

In still another embodiment, the presently described subject matter is directed to a method for treating cancer, further comprising administering to the subject an effective amount of at least one cancer chemotherapeutic agent.

In a further embodiment, the presently described subject matter is directed to a method for treating cancer, wherein the at least one cancer chemotherapeutic agent is administered simultaneous with, before or after administration of the pharmaceutical composition.

In yet a further embodiment, the presently described subject matter is directed to a method for treating cancer, further comprising administering to the subject radiation therapy.

In an embodiment, the presently described subject matter is directed to a method for treating cancer, wherein radiation therapy is administered simultaneous with, before or after administration of the pharmaceutical composition.

In a further embodiment, the presently described subject matter is directed to a method for treating RING E3 ubiquitin ligase related disease, wherein the RING E3 ubiquitin ligase related disease is an inflammatory disorder.

In another embodiment, the presently described subject matter is directed to a method for treating an inflammatory disorder, wherein the inflammatory disorder is selected from the group consisting of pulmonary fibrosis; ischaemic heart disease; Crohn's disease; dermatomyositis; diabetes mellitus; Guillain-Barre syndrome; hashimoto's disease; idiopathic thrombocytopenic purpura; mixed connective tissue disease; myasthenia gravis; narcolepsy; pemphigus vulgaris; pernicious anaemia; polymyositis; primary biliary cirrhosis; Sjogren's syndrome; temporal arteritis; ulcerative colitis; vasculitis; Wegener's granulomatosis; systemic lupus erythematosus; lupus nephritis; Goodpasture's syndrome; haemolytic anaemia; thyrotoxicosis; multiple sclerosis; scleroderma; asthma; rheumatoid arthritis; osteoarthritis; septicaemia; artherosclerosis; chronic renal disease; inflammatory bowel disease; vasculitis; peritonitis; giant papillary conjunctivitis; uveitis; seasonal allergic conjunctivitis; chronic prostatitis; glomerulonephritis; hypersensitivities; inflammatory bowel diseases; pelvic inflammatory disease; reperfusion injury; transplant rejection; Chediak-Higashi syndrome; chronic granulomatous disease; urinary tract inflammatory conditions; interstitial cystitis; ulcerative colitis; systemic sclerosis; dermatomyositis; polymyositis; and inclusion body myositis.

In an embodiment, the presently described subject matter is directed to a method for treating an inflammatory disorder, wherein the pharmaceutical composition further comprises at least one anti-inflammatory agent.

In another embodiment, the presently described subject matter is directed to a method for treating an inflammatory disorder, wherein the anti-inflammatory agent is selected from the group consisting of a corticosteroid; cortisol; aldosterone; hydrocortisone; hydrocortisone acetate; cortisone acetate; tixocortol pivalate; prednisolone; methylprednisolone; prednisone; triamcinolone acetonide; triamcinolone alcohol; mometasone; amcinonide; budesonide; desonide; flucinonide; fluocinolone acetonide; halcinonide; betamethasone; betamethasone sodium phosphate; dexamethasone; dexamethasone dodium phosphate; flucortolone; hydrocortisone-17-butyrate; hydro cortisone-17-valerate; aclometasone dipropionate; betamethasone valerate; betamethasone dipropionate; prednicarbate; clobetasone-17-butyrate; clobetasol-17-propionate; flucortolone caproate; fluocortolone pivalate; fluprednidene acetate; a non-steroidal anti-inflammatory; a cox-2 inhibitor; nimesulide; diclofenac; licofelone; aspirin; ibuprofen; naproxen; an immune selective anti-inflammatory derivative; phenylalanine-glutamine-glycine; an herb; Harpagophytum; hyssop; ginger; turmeric; Arnica Montana; willow bark; and cannabis.

In yet another embodiment, the presently described subject matter is directed to a method for treating an inflammatory disorder, further comprising administering to the subject an effective amount of at least one anti-inflammatory agent.

In still another embodiment, the presently described subject matter is directed to a method for treating an inflammatory disorder, wherein the at least one anti-inflammatory agent is administered simultaneous with, before or after administration of the pharmaceutical composition.

In a further embodiment, the presently described subject matter is directed to a method for treating RING E3 ubiquitin ligase related disease, wherein the RING E3 ubiquitin ligase related disease is an angiogenesis disorder.

In a further embodiment, the presently described subject matter is directed to a method for treating an angiogenesis disorder, wherein the angiogenesis disorder is selected from the group consisting of diabetic blindness; chronic inflammation; arthritis; age-related macular degeneration; retinopathy; rheumatoid arthritis; osteoarthritis; Crohn's disease; psoriasis; cancer; Alzheimer's disease; restenosis; pulmonary fibrosis; asthma; angiofibroma; neovascular glaucoma; arteriovenous malformations; nonunion fractures; lupus; a connective tissue disorder; Osler-Weber syndrome; atherosclerotic plaque; corneal graft neovascularization; pyogenic granuloma; retrolental fibroplasias; scleroderma; granulation, hemangioma; trachoma; hemophilic joints; peritoneal endometriosis; adiposity; and vascular adhesions.

In yet a further embodiment, the presently described subject matter is directed to a method for treating an angiogenesis disorder, wherein the pharmaceutical composition further comprises at least one angiogenesis inhibitor.

In still a further embodiment, the presently described subject matter is directed to a method for treating an angiogenesis disorder, wherein the at least one angiogenesis inhibitor is selected from the group consisting of bevacizumab, sunitinib, sorafenib, thalidomide, lenalidomide, panitumumab, cetuximab, and erlotinib.

In an embodiment, the presently described subject matter is directed to a method for treating RING E3 ubiquitin ligase related disease, wherein the RING E3 ubiquitin ligase related disease is an autoimmune disease.

In another embodiment, the presently described subject matter is directed to a method for an autoimmune disease, wherein the autoimmune disease is selected from the group consisting of Crohn's disease, dermatomyositis, diabetes mellitus, Guillain-Barre syndrome, hashimoto's disease, idiopathic thrombocytopenic purpura, mixed connective tissue disease, myasthenia gravis, narcolepsy, pemphigus vulgaris, pernicious anaemia, polymyositis, primary biliary cirrhosis, Sjogren's syndrome, temporal arteritis, ulcerative colitis, vasculitis, Wegener's granulomatosis, systemic lupus erythematosus, lupus nephritis, Goodpasture's syndrome, haemolytic anaemia, thyrotoxicosis, multiple sclerosis, and scleroderma.

In an embodiment, the presently described subject matter is directed to a method for an autoimmune disease, further comprising administering to the subject a therapeutically effective amount of at least one member selected from the group consisting of an anti-cancer agent, an angiogenesis inhibitor, and an anti-inflammatory agent.

In a further embodiment, the presently described subject matter is directed to a method for treating RING E3 ubiquitin ligase related disease, wherein the pharmaceutical composition further comprises an effective amount of at least one member selected from the group consisting of an anti-cancer agent, an angiogenesis inhibitor and an anti-inflammatory agent.

In yet a further embodiment, the presently described subject matter is directed to a method for treating cancer wherein the pharmaceutical composition further comprises an anti-cancer agent, an angiogenesis inhibitor, and optionally an anti-inflammatory agent.

### EXAMPLES

### Example 1

### Synthetic pathway of compound A(7006286)

### N-Acetylanthranilic acid (2)

10 g (73 mmol) of anthranilic acid (1) was mixed with 15 ml of acetic anhydride and 15 ml of acetic acid and kept at reflux for 2 hours. After cooling, the mixture was poured onto ice and the precipitate was filtered and recrystallized from 50% EtOH. Yield 9.3 g (80%).

### 2-Methyl-3-(3-nitrophenyl)-4-quinazolone (3)

In a 250 ml flask were placed 3.58 g (20 mmol) of N-acetylanthranilic acid (2), 2.76 g (20 mmol) of 3-nitroaniline and 35 ml of toluene. The mixture, while stirring, was treated dropwise with a solution of 920 mg (6.7 mmol) of phosphorous trichloride in 5 ml of toluene. Then the resulting suspension was stirred under reflux for 2 hours. After cooling, it was treated with 40 ml of a 10% sodium carbonate solution. After removing toluene by indirect steam distillation in vacuo, the solid was removed by filtration and recrystallized from methanol. Yield—3.25 g (58%).

### 2-[2-(4-Hydroxyphenyl)-vinyl]-3-(3-nitrophenyl)-4-quinazolone (Compound A)

A mixture of 2-methyl-3-(3-nitrophenyl)-4-quinazolone (3) (2.81 g, 10 mmol), 4-hydroxybenzaldehyde (1.34 g, 11 mmol) and 600 mg (7.3 mmol) of anhydrous sodium acetate was dissolved in 25 ml of acetic acid and kept at reflux for 10 hours. The precipitate formed upon cooling and was filtered, washed with boiling water and dried on air. The yield of bright yellow powder (mp 286-89) was 1.72 g (45%).

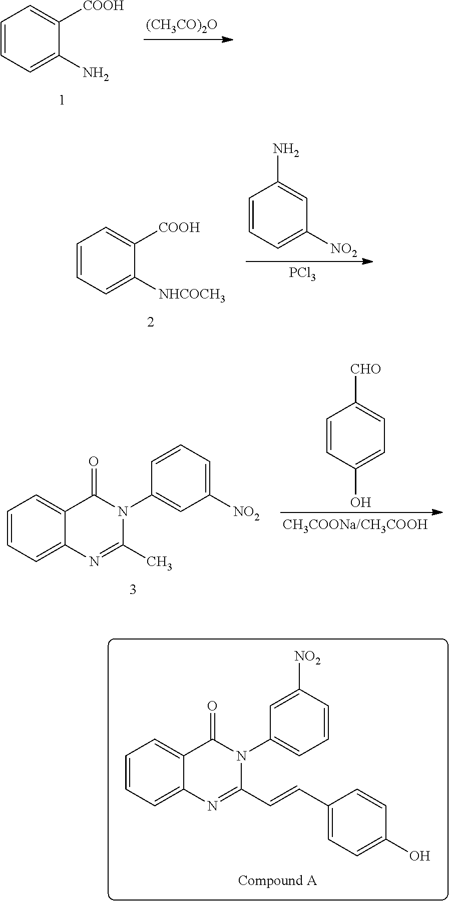

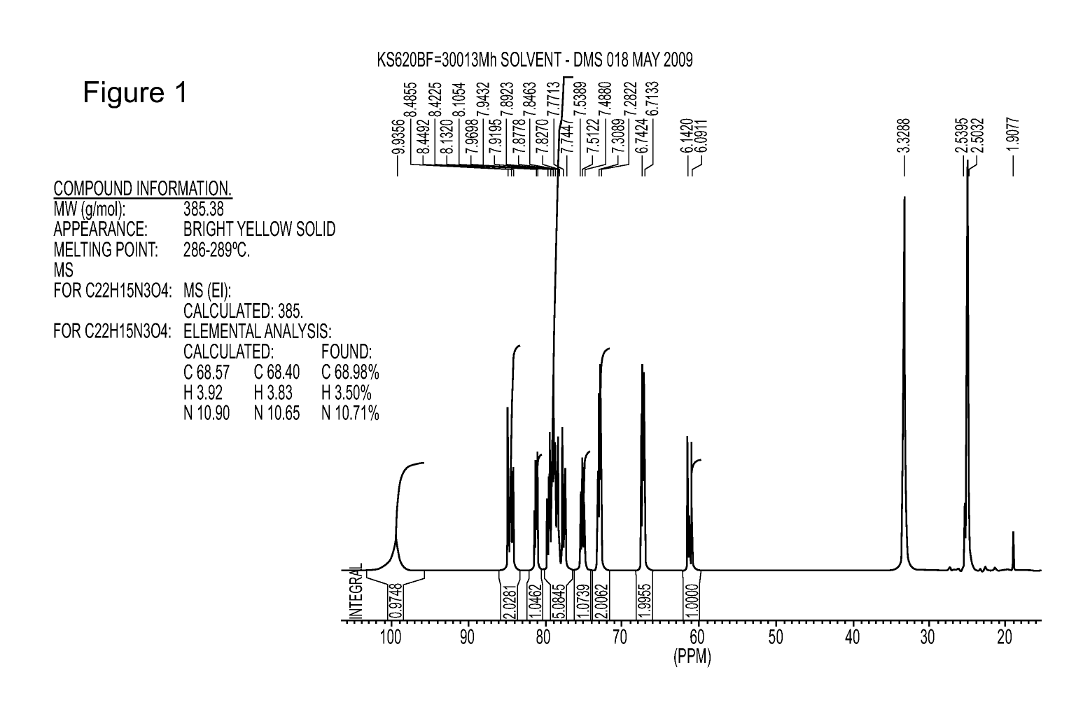

The characterization of Compound A via HNMR is shown in FIG. 1.

### Example 2

### Synthetic Pathway of Compound B (7084016)

### (E)-2-(4-methoxystyryl)-3-(3-nitrophenyl)quinazolin-4(3H)-one

A mixture of 2-methyl-3-(3-nitrophenyl)-3H-quinazolin-4-one (for synthesis 1 see preparation of Compound A 165 mg, 0.6 mmol), 4-methoxybenzaldheyde (120 mg, 0.70 mmol) and NaOAc (53 mg, 0.70 mmol) in glacial acetic acid (3.0 ml) was heated at reflux 10 hours. After the reaction mixture was allowed to cool to RT the resulting precipitate was collected by vacuum filtration, washed with water and recrystallized from EtOH to afford 114 mg (51%) of the target compound.

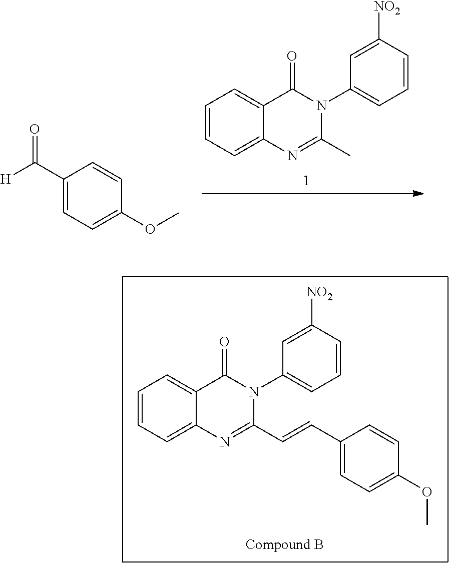

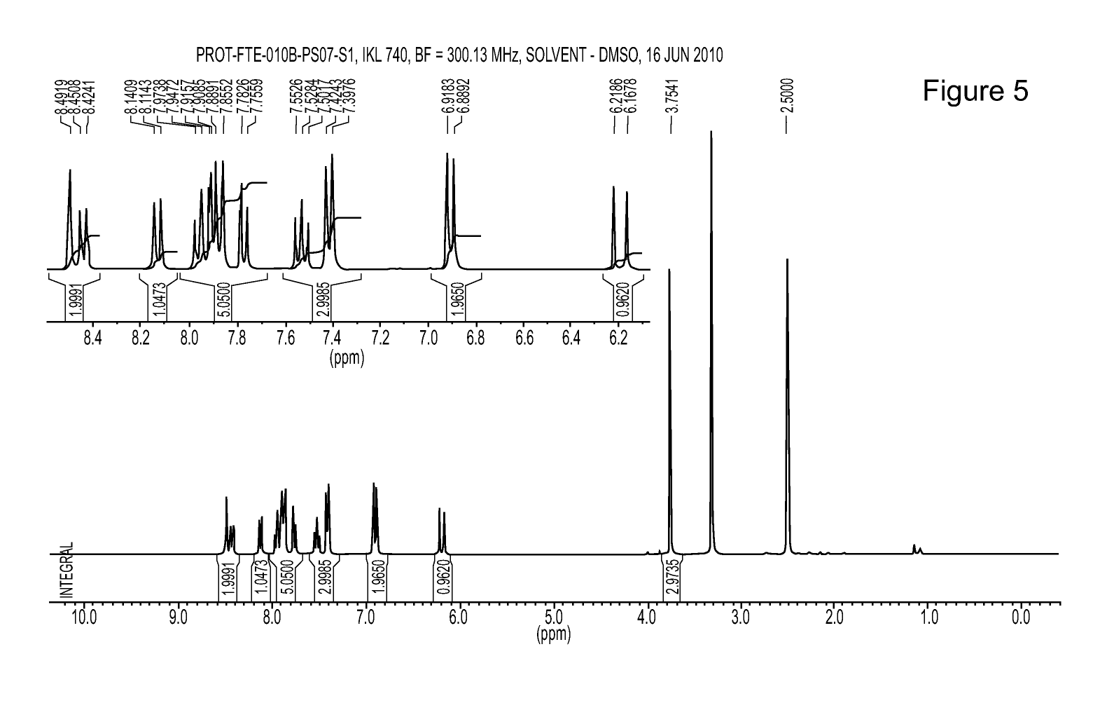

The characterization of Compound B via HNMR is shown in FIG. 5.

### Example 3

### Synthetic Pathway of Compound C (7084151)

### (E)-2-(4-bromostyryl)-3-(3-nitrophenyl)quinazolin-4(3H)-one

A mixture of 2-methyl-3-(3-nitrophenyl)-3H-quinazolin-4-one (for synthesis 1 see preparation of Compound A 165 mg, 0.6 mmol), 4-bromobenzaldheyde (120 mg, 0.70 mmol) and NaOAc (53 mg, 0.70 mmol) in glacial acetic acid (3.0 ml) was heated at reflux 10 hours. After the reaction mixture was allowed to cool to RT the resulting precipitate was collected by vacuum filtration, washed with water and recrystallized from EtOH to afford 114 mg (51%) of the target compound.

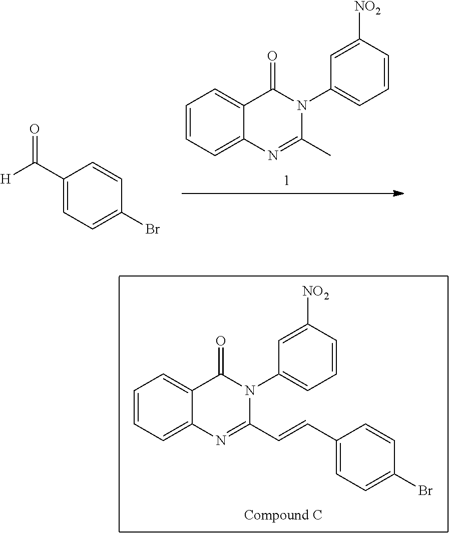

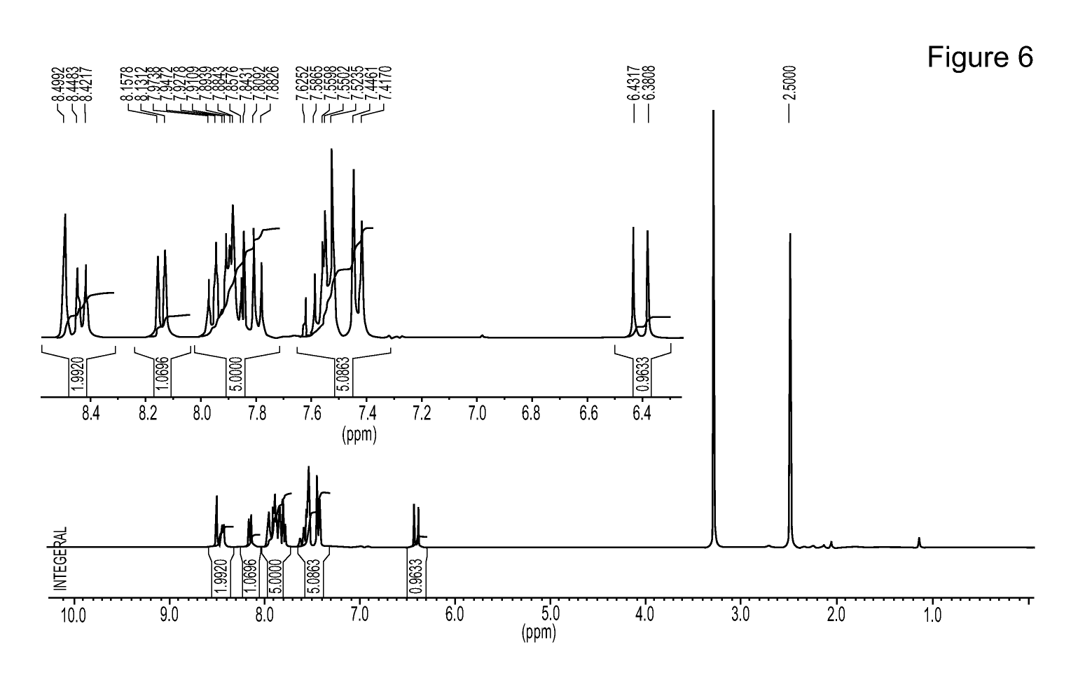

The characterization of Compound C via HNMR is shown in FIG. 6.

### Example 4

### Synthetic Pathway of Compound D (7084175)

### (E)-3-(3-(dihydroxyamino)phenyl)-2-(2-(1-methyl-1H-pyrazol-3-yl)vinyl)quinazolin-4(3H)-one

A mixture of 2-methyl-3-(3-nitrophenyl)-3H-quinazolin-4-one (for synthesis 1 see preparation of Compound A 165 mg, 0.6 mmol), 1-methyl-1H-pyrazole-3-carbaldehyde (120 mg, 0.70 mmol) and NaOAc (53 mg, 0.70 mmol) in glacial acetic acid (3.0 ml) was heated at reflux 10 hours. After the reaction mixture was allowed to cool to RT the resulting precipitate was collected by vacuum filtration, washed with water and recrystallized from EtOH to afford 114 mg (51%) of the target compound.

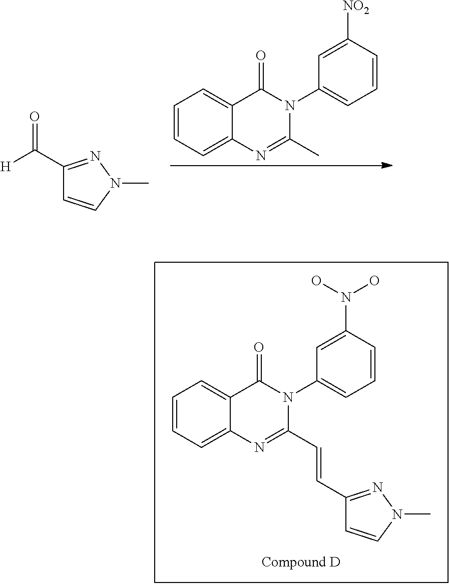

### Example 5

### In-Vitro Effect of Compounds A, B, C and Don Cancer Cells

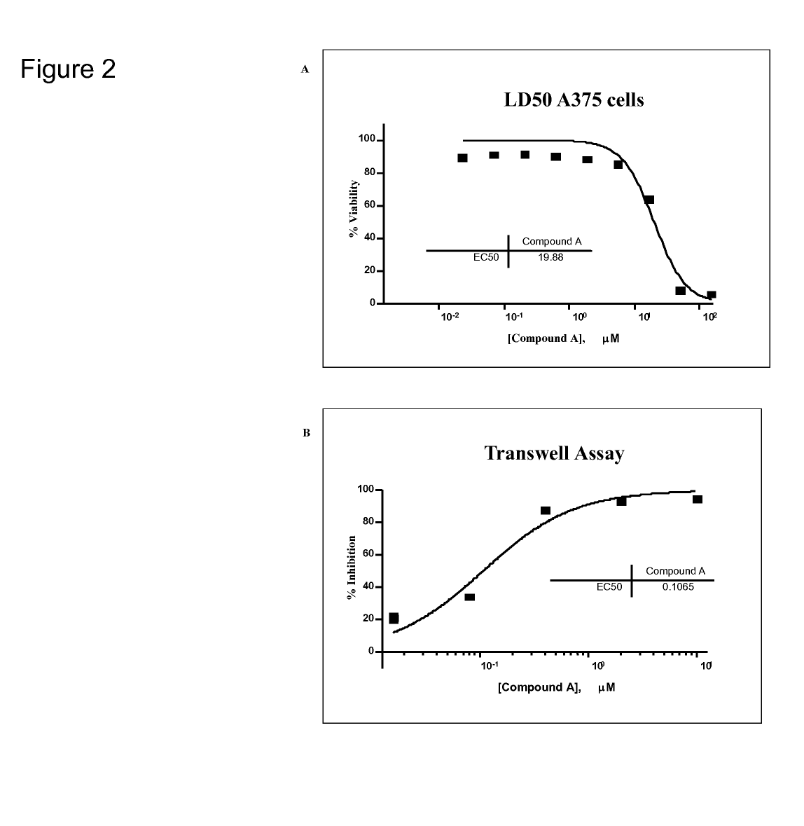

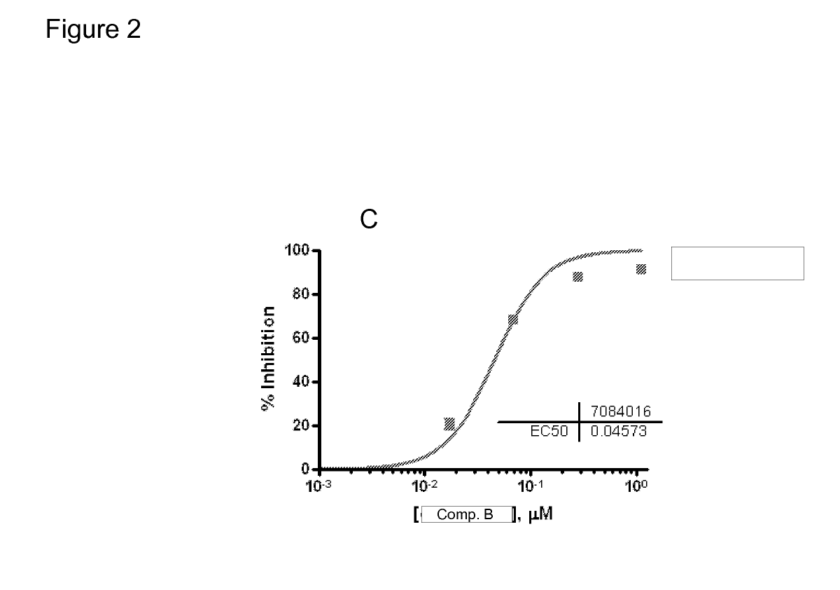

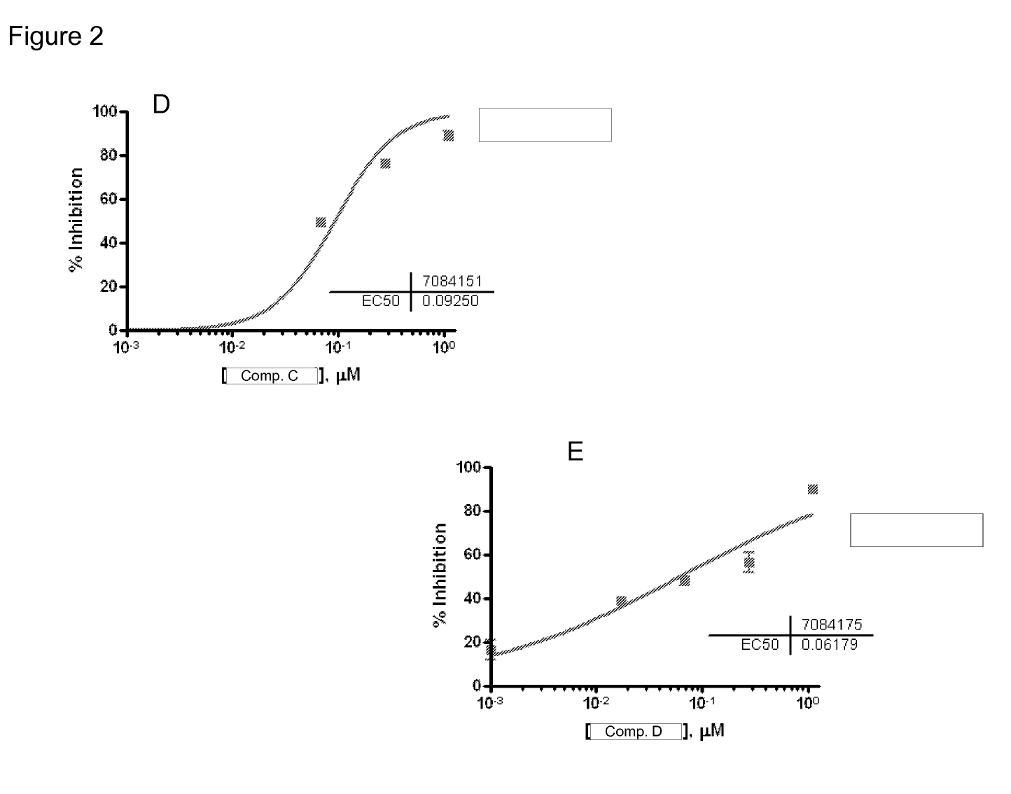

A375 cells (CRL-1619, Melanoma cancer cells, American Type Culture Collection (“ATCC”), Manassas Va., USA) were cultured with RPMI and 10% FBS (300 cells per well). Twenty-four hours after seeding the cells were treated with different concentrations of Compound A. Viability of the cells was tested seventy-two hours post treatment using WST-1 reagent (Roche). The LD50s were calculated using Prism software (Graphpad). Compound A killed the A375 cancer cells with LD50 of 20 μM (FIG. 2A).

The A375 cells were cultured with RPMI and 10% FBS. The cells were starved for 24 hours in medium without FBS. At the end of the starvation, 5×10⁴ cells in medium without FBS were placed in the upper chamber of 24-well, TRANSWELL apparatus (Corning TRANSWELL® polycarbonate membrane inserts, 5 μm pore size with solvent (DMSO/PEG400) or different concentrations of Compound A, B, C or D. Medium with FBS and compound A, B, C or D was added to the bottom chamber. Twenty-four hours later the cells from the upper chamber were removed with a cotton swab. Cells that migrated to the lower side of the membrane were stained with Calcein-AM (Sigma) and images were taken with a fluorescence microscope. The number of migrating cells was quantified by Image J software and the IC50 values were calculated using Prism software (Graphpad). FIG. 2B shows the inhibitory effect of Compound A on cell-migration with IC50 of 0.1 μM in A375 melanoma cell line.

The results from these experiments indicate that Compounds A, B, C and D have anti-migratory activity on A375 cells at a concentration which between 200 to 150-fold lower than the toxic concentration of these compounds. This indicates that these compounds can be used for indications involving cell migration without casing any significant cellular toxicity.

### Example 6

### In-Vivo Effect of Compound A Against Formation of Lung Cancer Metastasis

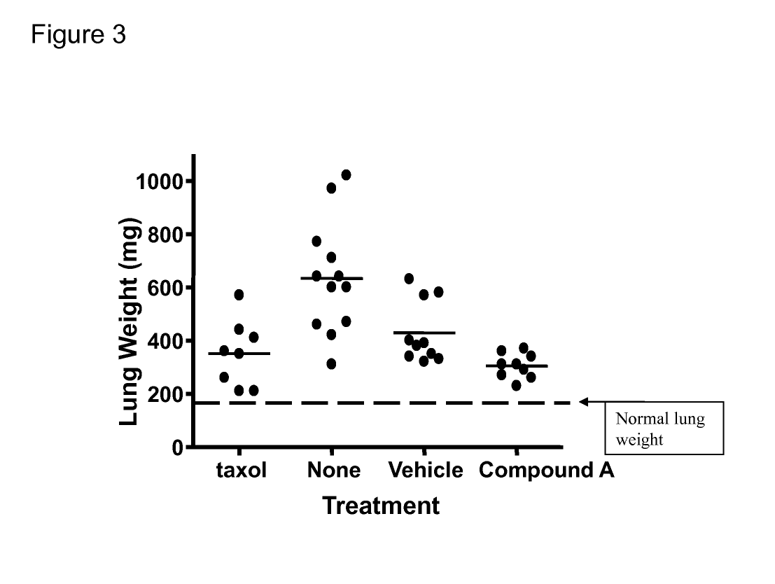

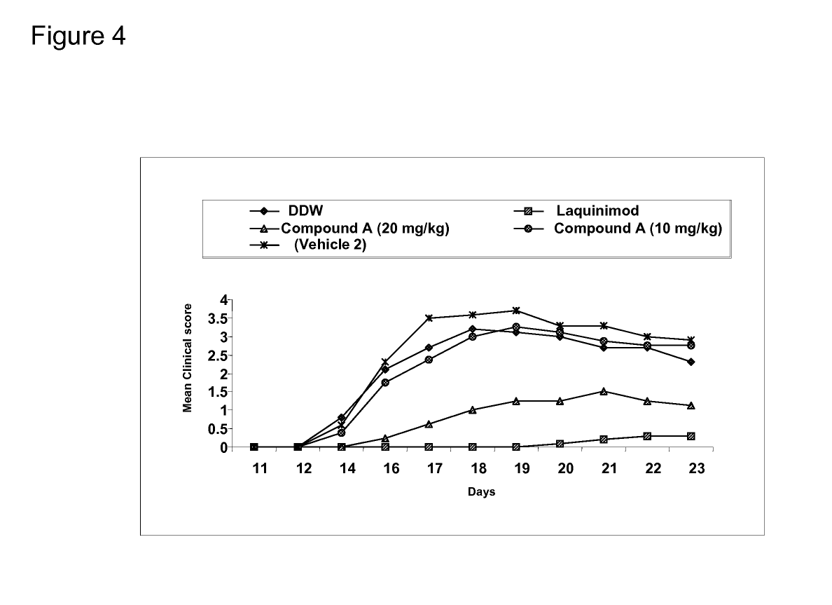

Suppressive activity against formation of lung cancer metastasis was tested using D122 Lewis lung cancer cells injected into the tail vein of male C57BL mice. Animals were treated twice daily by intraperitoneal injection of Compound A at 20 mg/kg or solvent (5% DMSO, 10% PEG400, 5% Solutol HS15, 5% Tween 80). Treatment was continued for 28 days. On day 33, animals were sacrificed, lungs were removed and weighed. When the Compound A and vehicle groups are compared, there is about a 50% decrease in added lung weight in the Compound A group (FIG. 3). The average weight gain in the control group was 250 mg while in the Compound A group it was only 125 mg. Analyzing the data using two tailed T test indicate a the data is significant (pValue=0.0085). The effect seen following administration of Compound A, supports the claim that this compound has anti-metastatic activity.

### Example 7

### Anti-Inflammatory Assay

A delayed-type hypersensitivity (DTH) reaction is an expression of cell-mediated immunity and plays a major role in the pathology and chronicity of many inflammatory disorders. Delayed-type hypersensitivity (DTH) reactions can be induced by various allergens, including oxazolone, 2,4-dinitrofluorobenzene (DNFB) and sheep red blood cells (SRBCs). One of the most characteristic DTH phenomena is contact hypersensitivity, which is characterized by swelling and by increased tissue levels of cytokines. Contact hypersensitivity (CHS) is a T cell-mediated immune reaction in response to cutaneous sensitization and challenge with reactive haptens that are capable of binding directly to soluble and cell-associated proteins and recognized by T cells in the context of self-MHC products. The cells that recognize antigen-protein complex in the skin are the Langerhans cells (LCs). After topically allergen application, Langerhans cells (LCs), the major antigen-presenting cells (APCs) for the induction of immune responses in skin, show enhanced expression of surface MHC class II molecules, and start to emigrate from the skin to regional lymph nodes where specific lymphocyte activation is thought to occur. After a second contact with the hapten, T cells are first recruited into tissues and then activated by antigen-presenting cells to produce cytokines that mediate local inflammation. Myeloperoxidase (MPO) is an enzyme exclusively present in neutrophil granules, which is commonly used as an index of granulocyte infiltration, and its inhibition is indicative of an anti-inflammatory action.

The goal of the present study is to examine the effects of Compounds A, B and C on oxazolone-induced DTH.

Animal:
Animal species and strain: 50 BALB/c mice
Sex and age and weight: Female, 19-21 g, 6-8 weeks
Breeder/supplier: Shanghai SLAC Laboratory Animal Co. Ltd.

Animal Quarters and Husbandry:
Test Facility: PharmaLegacy Laboratories Vivarium
Adaptation: Not less than 7 days
Room: Conventional Room
Room temperature: (19-26)° C.
Room relative humidity: 40-70%
Light cycle: Fluorescent light for 12-hour light (8:00-20:00) and
Food: Free access to food (irradiated, Shanghai SLAC Laboratory Animal Co. Ltd., China)
Water: Free access to water (municipal tap water filtered by

### Molanimal Ultrapure Water System)

Reagents:

Oxazolone: Sigma-Aldrich. (St. Louis, Mo., USA), Cat: E0753, Lot: 124K3690.

Pentobarbital sodium: Shanghai Westang Biotech Co., Ltd (Shanghai, P.R.China), Lot: WS20090520.

Isoflurane: He Bei Jiu Pai Biotech Co., Ltd, Cat: H19980141.

Acetone: Sinopharm Chemical Reagent Co., Ltd, Cat: 10000418.

Olive oil: Sinopharm Chemical Reagent Co., Ltd, Cat: 69018028.

Saline: Anhui double-crane Pharmaceutical Co., Ltd, Cat: H34023609.

Reference drug: Dexamethasone: Sigma-Aldrich, Cat: D1756-1G.

Equipment:

Micrometer: Mitutoyo (No. 045-020, 0-25 mm, 0.001 mm, Japan)

Procedures:

Reagents were setup as follows:

Oxazolone solution: Oxazolone was dissolved in 4:1 acetone/olive oil at 10 mg/mL.

Reference drug solution: Dexamethasone was dissolved in acetone at 2.5 mg/mL.

### Immunization and Challenge:

Randomized, 50 animals were broken into 5 groups (n=10).

Mice were anesthetized with 1.0% pentobarbital sodium (60 mg/kg), and their abdomens shaved. 150 μL 3% oxazolone in 4:1 acetone/olive oil was painted on abdomen of each mouse on day 0.

All the mice were challenged by applying 20 μL 1% oxazolone in 4:1 acetone/olive oil onto both sides of right ear topically (10 μL/side) on day 5.

### Treatment:

Compound A, B and C, and the reference drug were administered following different dosage protocols: a) Group 1 (vehicle group), saline was administrated orally 1 hour before Oxazolone challenge. b) Reference drug group, dexamethasone (0.05 mg/ear) in acetone, was applied topically (20 μL/ear, 10 μL/side) to both sides of right ear 1 hours, and 6 hours after Oxazolone challenge in Group 2. Different dosage of three test articles in saline were administered orally 1 hour before Oxazolone challenge in Group 3, 4, 5.

### Measurement:

Body weights of all mice were recorded daily starting from Day 0 to Day 6.

Ear swelling response was determined by ear thickness measured with a micrometer before and 24 hours after oxazolone challenge and reported as the mean change in ear thickness (ΔT±S.E.M.). Percent suppression of ear swelling response was calculated as % suppression=[1−(ΔT of sensitized mice exposed to experimental treatment/ΔT of sensitized mice exposed to vehicle treatment)]×100.

Mice were terminated by 95% CO₂ after the last ear thickness measurement (24 hours after Oxazolone challenge).

The ear pinnas of each group were collected immediately after the sacrifice by punching with a 10 mm diameter punch and weighed. For group 1 both left and right ears were collected, for group 2-5 only right ears were collected. The ear samples were frozen in liquid nitrogen.

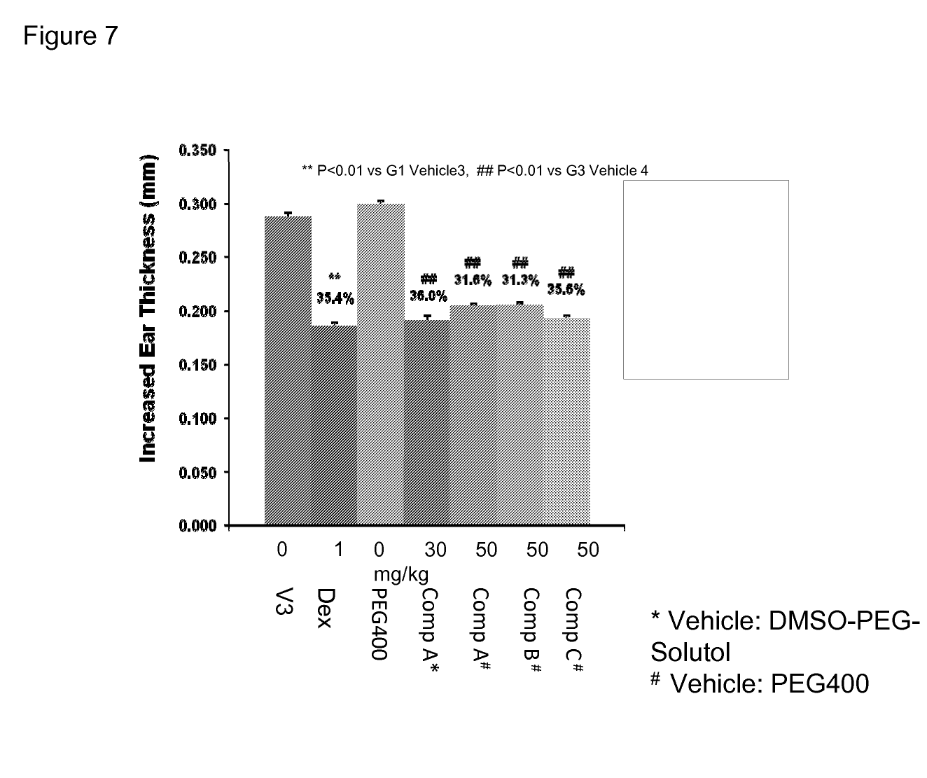

As shown in FIG. 7, Compounds A, B and C have the same anti-inflammatory activity as the control.

### Statistics:

Results were presented as mean±S.E.M. One-way analysis of variance followed by Dunnett's post test was used to determine differences between groups. P<0.05 was considered statistically significant.

### Example 8

### EAE Model for Multiple Sclerosis

The EAE model in CSJL/F1 mice is an established model for multiple sclerosis. Disease is induced in all mice by the injection of the encephalitogenic emulsion (MSCH/CFA, 0.05 ml injected into the left foot-pad of each mouse). Treatment with Compound A at 10 and 20 mg/kg or solvent (5% DMSO, 10% PEG400, 5% Solutol HS15, 5% Tween 80) by intraperitoneal injection, was started from day 1 of the study and administered twice daily in the first 9 days and then once daily until the end of the study. Scoring of EAE clinical signs were initiated from the 10^{th} day post-EAE induction and continued daily. The clinical signs were recorded according to a grading system described in the table 1.

TABLE 1

| Score | Signs | Description |
|---|---|---|
| 0 | Normal behavior | No neurological signs. |
| 1 | Tail weakness | The mouse tail is limp and droops. |
| 2 | Hind legs weakness | Limb paresis, wobbly walk-when the mouse |
| | | walks the hind legs is unsteady. |
| 3 | Hind legs paralysis | The mouse can't move its hind legs |
| | | and it drags them when he walks. |
| 4 | Full paralysis | The mouse can't move its legs at all, it looks |
| | | thinner and emaciated. |
| 5 | Death | |

Administration of Compound A at 20 mg/kg diminished the clinical signs induced by the induction of EAE to about half of the score for water and vehicle controls. No effect was observed when Compound A was administered at 10 mg/kg, indicating that an effective concentration was not achieved at this dosing regime. A group treated with Laquinimod, supplied by Teva Pharmaceutical Industries, (1 mg/kg daily oral administration) served as a positive control.

### Example 9

### Use of Compounds A, B, C and D

A patient is suffering from breast cancer. A therapeutically effective amount of compound A, B, C or D is administered to the patient in an acceptable dosage form. It would be expected that the patient would improve his/her condition or recover.

Throughout this specification, various scientific publications and U.S. patents or U.S. published patent applications are referenced. The entire disclosure of each of these publications is hereby incorporated by reference herein in their entirety. Citation or identification of any reference in this section or any other part of this application shall not be construed as an admission that such reference is available as prior art to the invention.

It will be appreciated by those skilled in the art to which the present subject matter pertains that various modifications can be made without departing from the essential nature thereof. It is intended to encompass all such modification within the scope of the appended claims.

We claim:

1. A method for treating a RING E3 ubiquitin ligase related disease in a subject, comprising administering to the subject a therapeutically effective amount of a compound of general formula I 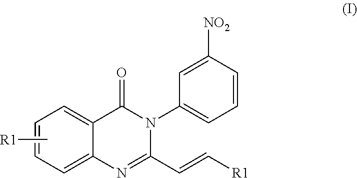
wherein:
R1 is H, C₁₋₆ alkyl, C₁₋₆ alkenyl, C₁₋₆ alkynyl, arylalkyl, cyano, hydroxyl, alkoxyl, hydroxyalkyl, or alkoxyalkyl, aryl or heteroaryl, optionally substituted by 1-5 R₃;
each R₃ is independently selected from halogen, hydroxy, hydroxyalkyl, hydroxyalkoxy, C₁-C₁₀ alkyl, C₁-C₆ haloalkyl, C₁-C₁₀ alkoxy, alkoxyalkyl, alkoxyalkoxy, C₁-C₆ haloalkoxy, C₆-C₁₀ aryl, C₄-C₁₀ heteroaryl, C₇-C₁₂ aralkyl, C₅-C₁₂ heteroaralkyl, C₂-C₈ heterocyclyl, C₂-C₁₂ alkenyl, C₂-C₁₂ alkynyl, C₅-C₁₀ cycloalkenyl, C₄-C₁₀ heterocycloalkenyl, carboxy, carboxylate, cyano, nitro, amino, aminoalkyl, C₁-C₆ alkylamino, C₁-C₆ alkylaminoalkyl, C₁-C₆ dialkylamino, C₁-C₆ dialkylaminoalkyl, mercapto, SO₃H, —S— alkyl, —S(O)alkyl, S(O)₂alkyl, sulfate, S(O)NH₂, S(O)₂NH₂, phosphonyl, acyl, aminocarbonyl, C₁-C₆ alkylaminocarbonyl, C₁-C₆ dialkylaminocarbonyl, C₁-C₁₀ alkoxycarbonyl, thioalkoxycarbonyl, hydrazinocarbonyl, C₁-C₆ alkyl hydrazinocarbonyl, C₁-C₆ dialkyl hydrazinocarbonyl, hydroxyaminocarbonyl, alkoxyaminocarbonyl, —NHSO₂NH₂, or —OCH₂CH₂N(R₇)₂;
R2 is H, C₁₋₆ alkyl, C₁₋₆ alkenyl, C₁₋₆ alkynyl, arylalkyl, cyano, hydroxyl, alkoxyl, hydroxyalkyl, or alkoxyalkyl, aryl or heteroaryl, optionally substituted by 1-5 R₃;
each R₃ is as described above;
or an enantiomer or pharmaceutically acceptable salt thereof.

2. A method for treating a RING E3 ubiquitin ligase related disease in a subject, comprising administering to the subject a therapeutically effective amount of a pharmaceutical composition comprising a compound of general formula I 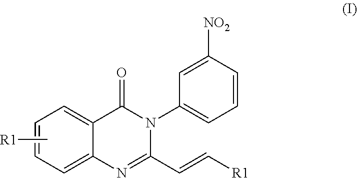
wherein:
R1 is H, C₁₋₆ alkyl, C₁₋₆ alkenyl, C₁₋₆ alkynyl, arylalkyl, cyano, hydroxyl, alkoxyl, hydroxyalkyl, or alkoxyalkyl, aryl or heteroaryl, optionally substituted by 1-5 R₃;
each R₃ is independently selected from halogen, hydroxy, hydroxyalkyl, hydroxyalkoxy, C₁-C₁₀ alkyl, C₁-C₆ haloalkyl, C₁-C₁₀ alkoxy, alkoxyalkyl, alkoxyalkoxy, C₁-C₆ haloalkoxy, C₆-C₁₀ aryl, C₄-C₁₀ heteroaryl, C₇-C₁₂ aralkyl, C₅-C₁₂ heteroaralkyl, C₂-C₈ heterocyclyl, C₂-C₁₂ alkenyl, C₂-C₁₂ alkynyl, C₅-C₁₀ cycloalkenyl, C₄-C₁₀ heterocycloalkenyl, carboxy, carboxylate, cyano, nitro, amino, aminoalkyl, C₁-C₆ alkylamino, C₁-C₆ alkylaminoalkyl, C₁-C₆ dialkylamino, C₁-C₆ dialkylaminoalkyl, mercapto, SO₃H, —S— alkyl, —S(O)alkyl, S(O)₂alkyl, sulfate, S(O)NH₂, S(O)₂NH₂, phosphonyl, acyl, aminocarbonyl, C₁-C₆ alkylaminocarbonyl, C₁-C₆ dialkylaminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, alkoxyaminocarbonyl, —NHSO₂NH₂, or —OCH₂CH₂N(R₇)₂;
R2 is H, C₁₋₆ alkyl, C₁₋₆ alkenyl, C₁₋₆ alkynyl, arylalkyl, cyano, hydroxyl, alkoxyl, hydroxyalkyl, or alkoxyalkyl, aryl or heteroaryl, optionally substituted by 1-5 R₃;
each R₃ is as described above;
or an enantiomer or pharmaceutically acceptable salt thereof.

3. The method according to claim 2, wherein the compound is selected from the group consisting of:
compound A of the formula 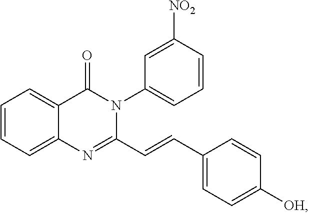
compound B of the formula 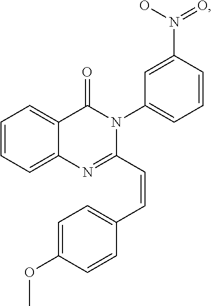
compound C of the formula 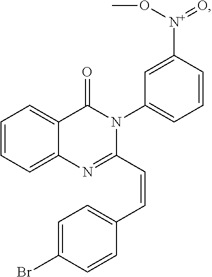
compound D of the formula

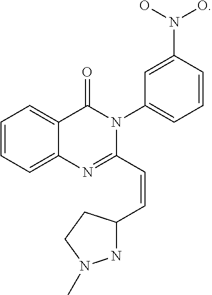

4. The method according to claim 2, wherein the pharmaceutical composition further comprises at least one pharmaceutically acceptable carrier.

5. The method according to claim 1, wherein the RING E3 ubiquitin ligase related disease is selected from the group consisting of cancer, an inflammatory disorder, an autoimmune disease and an angiogenesis disorder.

6. The method according to claim 5, wherein the RING E3 ubiquitin ligase related disease is a cancer.

7. The method according to claim 6, wherein the cancer is selected from the group consisting of anal cancer, astrocytoma, leukemia, lymphoma, head and neck cancer, liver cancer, testicular cancer, cervical cancer, sarcoma, hemangioma, esophageal cancer, eye cancer, laryngeal cancer, mouth cancer, mesothelioma, myeloma, oral cancer, rectal cancer, throat cancer, bladder cancer, breast cancer, uterine cancer, ovarian cancer, prostate cancer, lung cancer, colon cancer, pancreatic cancer, renal cell carcinoma, gastric cancer, skin cancer, basal cell carcinoma, melanoma, squamous cell carcinoma, oral squamous cell carcinoma, colorectal cancer, glioblastoma multiforme, endometrial cancer and malignant glioma.

8. The method according to claim 6, wherein the pharmaceutical composition further comprises an effective amount of at least one anti-cancer agent.

9. The method according to claim 8, wherein the at least one anti-cancer agent is selected from the group consisting of imatinib, dasatinib, axitinib, bosutinib, cediranib, erlotinib, gefitinib, lapatinib, lestaurtinib, nilotinib, semaxanib, sunitinib, toceranib, vandetanib, and vatalanib.

10. The method according to claim 6, further comprising administering to the subject an effective amount of at least one anti-cancer agent.

11. The method according to claim 10, wherein the at least one anti-cancer agent is administered simultaneous with, before or after administration of the pharmaceutical composition.

12. The method according to claim 6, wherein the pharmaceutical composition further comprises an effective amount of at least one cancer chemotherapeutic agent.

13. The method according to claim 12, wherein the at least one cancer chemotherapeutic agent is selected from the group consisting of mechlorethamine, cyclophosphamide, ifosfamide, melphalan, chlorambucil, dicarbazine, streptazocine; carmustine, lomustine, semustine, chlorozotocin, busulfan, triethylenemelamine; thiotepa; hexamethylmelamine, an antimetabolite; methotrexate, fluorouracil, 5-fluorouracil, floxuridine (5′-fluoro-2′-deoxyuridine), idoxuridine, cytarabine; N-phosphonoacetyl-L-aspartate, 5-azacytidine, azaribine, 6-azauridine, pyrazofuran, 3-deazauridine, acivicin, a purine analog, thioguanine, mercaptopurine, azathioprine, pentostatin, erythrohydroxynonyladenine, a vinca alkaloid, vincristine, vinblastine, an epipodophyllotoxin, etoposide, teniposide, an antibiotic, dactinomycin, daunorubicin, doxorubicin, bleomycin sulfate, plicamycin, mitomycin, an enzyme, L-asparaginase, a platinum coordination complex, cisplatin, carboplatin, hydroxyurea, procarbazine, mitotane, a hormone, an adrenocorticosteroid, prednisone, prednisolone, aminoglutethimide, a progestin, hydroxyprogesterone caproate, medroxyprogesterone acetate, megesterol acetate, estrogen, an androgen, diethylstilbestrol, fluoxymesterone, ethynyl estradiol, an antiestrogen, tamoxifen, a gonadotropin-releasing hormone analog, and leuprolide.

14. The method according to claim 6, further comprising administering to the subject an effective amount of at least one cancer chemotherapeutic agent.

15. The method according to claim 14, wherein the at least one cancer chemotherapeutic agent is administered simultaneous with, before or after administration of the pharmaceutical composition.

16. The method according to claim 6, further comprising administering to the subject radiation therapy.

17. The method according to claim 16, wherein radiation therapy is administered simultaneous with, before or after administration of the pharmaceutical composition.

18. The method according to claim 5, wherein the RING E3 ubiquitin ligase related disease is an inflammatory disorder.

19. The method according to claim 18, wherein the inflammatory disorder is selected from the group consisting of pulmonary fibrosis, ischaemic heart disease, Crohn's disease, dermatomyositis, diabetes mellitus, Guillain-Barre syndrome, hashimoto's disease, idiopathic thrombocytopenic purpura, mixed connective tissue disease, myasthenia gravis, narcolepsy, pemphigus vulgaris, pernicious anaemia, polymyositis, primary biliary cirrhosis, Sjogren's syndrome, temporal arteritis, ulcerative colitis, vasculitis, Wegener's granulomatosis, systemic lupus erythematosus, lupus nephritis, Goodpasture's syndrome, haemolytic anaemia, thyrotoxicosis, multiple sclerosis, scleroderma, asthma, rheumatoid arthritis, osteoarthritis, septicaemia, artherosclerosis, chronic renal disease, inflammatory bowel disease, vasculitis, peritonitis, giant papillary conjunctivitis, uveitis, seasonal allergic conjunctivitis, chronic prostatitis, glomerulonephritis, hypersensitivities, inflammatory bowel diseases, pelvic inflammatory disease, reperfusion injury, transplant rejection, Chediak-Higashi syndrome, chronic granulomatous disease, urinary tract inflammatory conditions, interstitial cystitis, ulcerative colitis, systemic sclerosis, dermatomyositis, polymyositis, and inclusion body myositis.

20. The method according to claim 18, wherein the pharmaceutical composition further comprises at least one anti-inflammatory agent.

21. The method according to claim 20, wherein the anti-inflammatory agent is selected from the group consisting of a corticosteroid, cortisol, aldosterone, hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, flucinonide, fluocinolone acetonide, halcinonide, betamethasone, betamethasone sodium phosphate, dexamethasone, dexamethasone dodium phosphate, flucortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, flucortolone caproate, fluocortolone pivalate, fluprednidene acetate, a non-steroidal anti-inflammatory, a cox-2 inhibitor, nimesulide, diclofenac, licofelone, aspirin, ibuprofen, naproxen, an immune selective anti-inflammatory derivative, phenylalanine-glutamine-glycine, an herb, Harpagophytum, hyssop, ginger, turmeric, Arnica Montana, willow bark and cannabis.

22. The method according to claim 18, further comprising administering to the subject an effective amount of at least one anti-inflammatory agent.

23. The method according to claim 22, wherein the at least one anti-inflammatory agent is administered simultaneous with, before or after administration of the pharmaceutical composition.

24. The method according to claim 5, wherein the RING E3 ubiquitin ligase related disease is an angiogenesis disorder.

25. The method according to claim 24, wherein the angiogenesis disorder is selected from the group consisting of diabetic blindness, chronic inflammation, arthritis, age-related macular degeneration, retinopathy, rheumatoid arthritis, osteoarthritis, Crohn's disease, psoriasis, cancer, Alzheimer's disease, restenosis, pulmonary fibrosis, asthma, angiofibroma, neovascular glaucoma, arteriovenous malformations, nonunion fractures, lupus, a connective tissue disorder, Osler-Weber syndrome, atherosclerotic plaque, corneal graft neovascularization, pyogenic granuloma, retrolental fibroplasias, scleroderma, granulation, hemangioma, trachoma, hemophilic joints, peritoneal endometriosis, adiposity and vascular adhesions.

26. The method according to claim 24, wherein the pharmaceutical composition further comprises at least one angiogenesis inhibitor.

27. The method according to claim 26, wherein the at least one angiogenesis inhibitor is selected from the group consisting of bevacizumab, sunitinib, sorafenib, thalidomide, lenalidomide, panitumumab, cetuximab, and erlotinib.

28. The method according to claim 5, wherein the RING E3 ubiquitin ligase related disease is an autoimmune disease.

29. The method according to claim 28, wherein the autoimmune disease is selected from the group consisting of Crohn's disease, dermatomyositis, diabetes mellitus, Guillain-Barre syndrome, hashimoto's disease, idiopathic thrombocytopenic purpura, mixed connective tissue disease, myasthenia gravis, narcolepsy, pemphigus vulgaris, pernicious anaemia, polymyositis, primary biliary cirrhosis, Sjogren's syndrome, temporal arteritis, ulcerative colitis, vasculitis, Wegener's granulomatosis, systemic lupus erythematosus, lupus nephritis, Goodpasture's syndrome, haemolytic anaemia, thyrotoxicosis, multiple sclerosis, and scleroderma.

30. The method according to claim 2, further comprising administering to the subject a therapeutically effective amount of at least one member selected from the group consisting of an anti-cancer agent, an angiogenesis inhibitor, and an anti-inflammatory agent.

31. The method according to claim 2, wherein the pharmaceutical composition further comprises an effective amount of at least one member selected from the group consisting of an anti-cancer agent, an angiogenesis inhibitor and an anti-inflammatory agent.

32. The method according to claim 6, wherein the pharmaceutical composition further comprises an anti-cancer agent, an angiogenesis inhibitor, and optionally an anti-inflammatory agent.

33. A compound of formula II 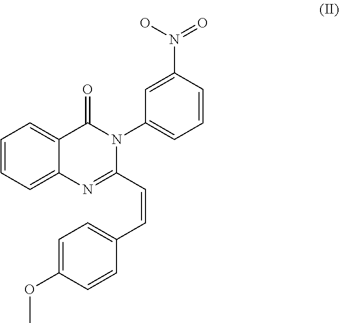
or a pharmaceutically acceptable salt thereof.

34. A compound of formula III 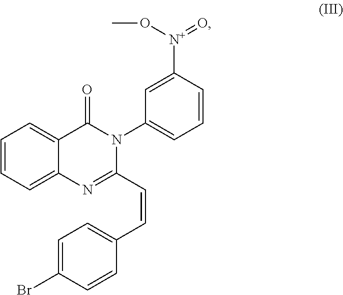
or a pharmaceutically acceptable salt thereof.

35. A compound of formula IV 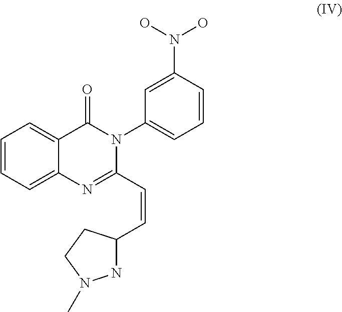
or a pharmaceutically acceptable salt thereof.
